(19) United States
(12) Patent Application Publication
(10) Pub. No.: US 2022/0062589 A1
(43) Pub. Date: Mar. 3, 2022

(54) Catheter Stabilizing Device

(71) Applicants: Maria Rodriguez, Visalia, CA (US); Deborah Robinson, Visalia, CA (US)

(72) Inventors: Maria Rodriguez, Visalia, CA (US); Deborah Robinson, Visalia, CA (US)

(21) Appl. No.: 17/009,058

(22) Filed: Sep. 1, 2020

(51) Int. Cl. A61M 25/02; A61M 1/28

(52) U.S. Cl. CPC A61M 25/02; A61M 2210/1021; A61M 2025/0266; A61M 1/285

(57) ABSTRACT

A catheter stabilizing device for stabilizing a Continuous Ambulatory Peritoneal Dialysis (CAPD) catheter includes a pad having an adhesive engaged to a first face thereof. The adhesive is used to engage the pad to abdominal skin of a user so that the pad is engaged to an abdomen of the user. A connector is engaged to a second face of the pad and can selectively connect to a tube of a catheter. The connector stabilizes the catheter in a selected position.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to stabilizing devices and more particularly pertains to a new stabilizing device for stabilizing a Continuous Ambulatory Peritoneal Dialysis catheter.

### (2) Description of Related Art Including Information Disclosed Under 37 CFR 1.97 and 1.98

The prior art relates to stabilizing devices, and more particularly to stabilizing devices intended for stabilization of Continuous Ambulatory Peritoneal Dialysis catheters. Prior art stabilizing devices may comprise belts or straps that encircle the abdomen, vests or harnesses positionable over the abdomen, and adhesively attached pads having clamping devices attached thereto.

### BRIEF SUMMARY OF THE INVENTION

An embodiment of the disclosure meets the needs presented above by generally comprising a pad having an adhesive engaged to a first face thereof. The adhesive is configured to adhesively engage the pad to abdominal skin of a user so that the pad is engaged to an abdomen of the user. A connector is engaged to a second face of the pad and is configured to selectively connect to a tube of a catheter. The connector is configured to stabilize the catheter in a selected position.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a front isometric perspective view of a catheter stabilizing device according to an embodiment of the disclosure.

FIG. 2 is a rear isometric perspective view of an embodiment of the disclosure.

FIG. 3 is a front in-use view of an embodiment of the disclosure.

FIG. 4 is a cross-sectional view of an embodiment of the disclosure.

FIG. 5 is an in-use view of an embodiment of the disclosure.

### DETAILED DESCRIPTION OF THE INVENTION

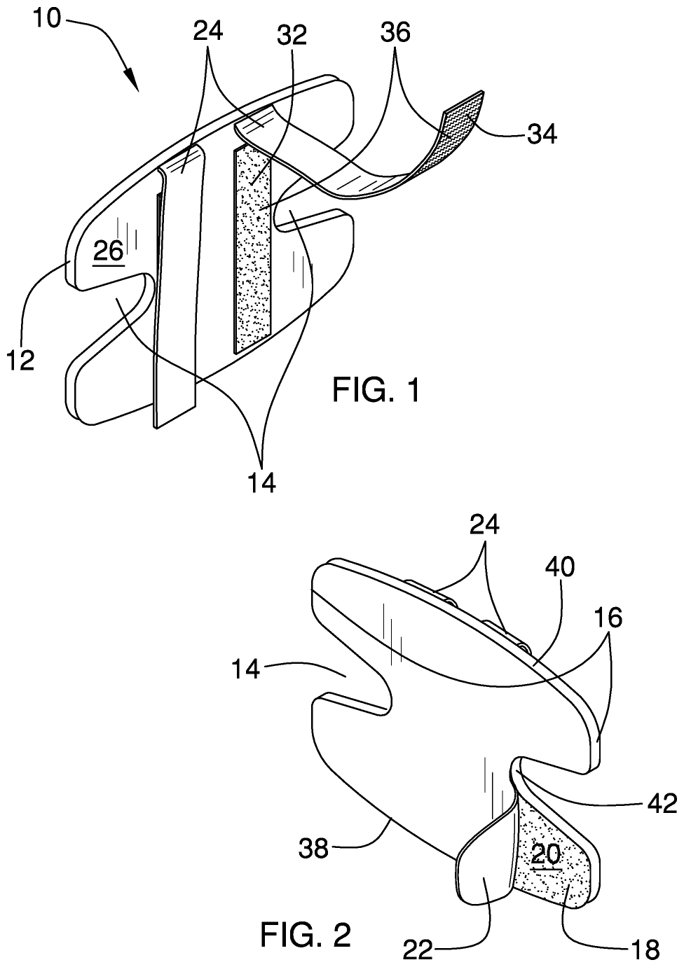

With reference now to the drawings, and in particular to FIGS. 1 through 5 thereof, a new stabilizing device embodying the principles and concepts of an embodiment of the disclosure and generally designated by the reference numeral 10 will be described.

As best illustrated in FIGS. 1 through 5, the catheter stabilizing device 10 generally comprises a pad 12, which is substantially ovally shaped. The pad 12 has a pair of cutouts 14 extending arcuately and singly thereinto from opposed ends 16 thereof. The cutouts 14 are configured to facilitate conforming of the pad 12 to contours of an abdomen of a user.

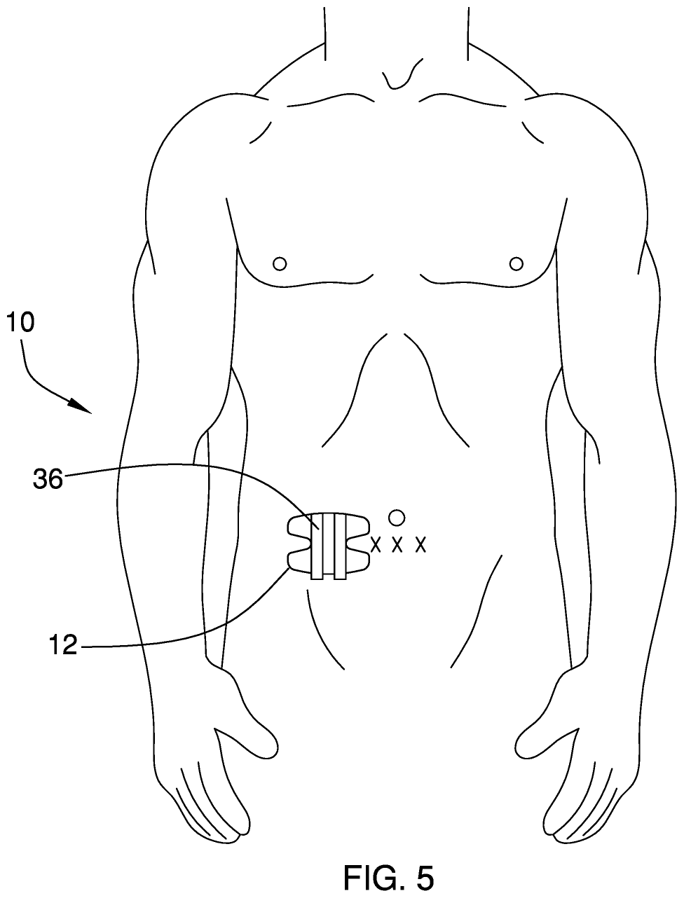

The pad 12 has an adhesive 18 engaged to a first face 20 thereof. The adhesive 18 is configured to adhesively engage the pad 12 to abdominal skin of a user so that the pad 12 is engaged to an abdomen of the user, as shown in FIG. 5.

A sheet 22 is removably engaged to the adhesive 18. The sheet 22 is positioned to prevent inadvertent adhesion of the adhesive 18. The sheet 22 is configured for removal from the adhesive 18, positioning the adhesive 18 to adhesively engage the pad 12 to the abdominal skin of the user.

A connector 24 is engaged to a second face 26 of the pad 12 and is configured to selectively connect to a tube 28 of a catheter 30, more particularly to a tube 28 of a Continuous Ambulatory Peritoneal Dialysis (CAPD) catheter 30. The connector 24 is configured to stabilize the catheter 30 in a selected position.

The connector 24 comprises a first element 32 and a second element 34 of a hook and loop fastener 36. The first element 32 is engaged to the second face 26 of the pad 12 and extends from proximate to a bottom 38 to proximate to a top 40 of the pad 12. The second element 34 is engaged to the pad 12 proximate to the top 40 and extends therefrom. The second element 34 is configured to be positioned over the tube 28 of a catheter 30 that is positioned upon the first element 32. The second element 34 is positioned to engage the first element 32 so that the hook and loop fastener 36 is configured to stabilize the catheter 30 in the selected position.

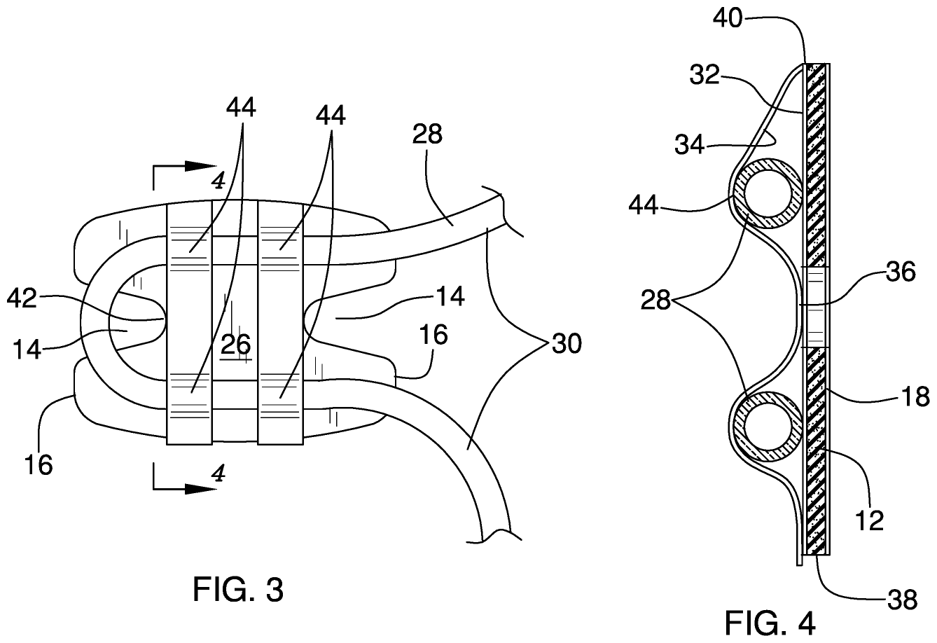

The connector 24 is one of a pair of connectors 24, with each connector 24 being positioned proximate to an inner limit 42 of an associated cutout 14. Each connector 24 is configured to selectively connect to a respective site 44 along the tube 28 of the catheter 30 so that the tube 28 extends loopedly across the second face 26 of the pad 12, as shown in FIG. 3.

In use, the sheet 22 is pealed from the adhesive 18 and the pad 12 is adhesively engaged to the abdomen of the user. The hook and loop fasteners 36 are utilized to stabilize the tube 28 of the catheter 30 in the desired position. Use of the device 10 is anticipated to reduce use of improvised measures to stabilize catheters 30, such as taping the tube 28 of the catheter 30 to the user, which can damage both the skin of the user and the tube 28. The device 10 effectively stabilizes the catheter 30, reducing a probability of the catheter 30 becoming dislodged or migrating to another position, which can cause contamination of the catheter 30.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the elements is present, unless the context clearly requires that there be only one of the elements.

I claim:

1. A catheter stabilizing device comprising:
a pad;
an adhesive engaged to a first face of the pad, wherein the adhesive is configured for adhesively engaging the pad to abdominal skin of a user, such that the pad is engaged to an abdomen of the user; and
a connector engaged to a second face of the pad and being configured for selectively connecting to a tube of a catheter, wherein the connector is configured for stabilizing the catheter in a selected position.

2. The catheter stabilizing device of claim 1, wherein the pad is substantially ovally shaped.

3. The catheter stabilizing device of claim 1, further including the pad having a pair of cutouts extending arcuately and singly thereinto from opposed ends thereof, wherein the cutouts are configured for facilitating conforming of the pad to contours of the abdomen of the user.

4. The catheter stabilizing device of claim 3, further including the connector being one of a pair of connectors, each connector being positioned proximate to an inner limit of an associated cutout, wherein each connector is configured for selectively connecting to a respective site along the tube of the catheter, such that the tube extends loopedly across the second face of the pad.

5. The catheter stabilizing device of claim 1, wherein the connector comprises a first element and a second element of a hook and loop fastener, the first element being engaged to the second face of the pad and extending from proximate to a bottom to proximate to a top of the pad, the second element being engaged to the pad proximate to the top and extending therefrom, wherein the second element is configured for positioning over the tube of a catheter positioned upon the first element, positioning the second element for engaging the first element, wherein the hook and loop fastener is configured for stabilizing the catheter in the selected position.

6. The catheter stabilizing device of claim 1, further including a sheet removably engaged to the adhesive, such that the sheet is positioned for preventing inadvertent adhesion of the adhesive, wherein the sheet is configured for removal from the adhesive, positioning the adhesive for adhesively engaging the pad to the abdominal skin of the user.

7. A catheter and a catheter stabilizing device combination comprising:
a catheter operationally engaged to a user;
a pad;
an adhesive engaged to a first face of the pad, wherein the adhesive is configured for adhesively engaging the pad to abdominal skin of a user, such that the pad is engaged to an abdomen of the user; and
a connector engaged to a second face of the pad, the connector being positioned for selectively connecting to a tube of a catheter for stabilizing the catheter in a selected position.

8. The catheter and a catheter stabilizing device combination of claim 7, wherein the pad is substantially ovally shaped.

9. The catheter and a catheter stabilizing device combination of claim 7, further including the pad having a pair of cutouts extending arcuately and singly thereinto from opposed ends thereof, wherein the cutouts are configured for facilitating conforming of the pad to contours of the abdomen of the user.

10. The catheter and a catheter stabilizing device combination of claim 9, further including the connector being one of a pair of connectors, each connector being positioned proximate to an inner limit of an associated cutout, such that each connector is positioned for selectively connecting to a respective site along the tube of the catheter, such that the tube extends loopedly across the second face of the pad.

11. The catheter and a catheter stabilizing device combination of claim 7, wherein the connector comprises a first element and a second element of a hook and loop fastener, the first element being engaged to the second face of the pad and extending from proximate to a bottom to proximate to a top of the pad, the second element being engaged to the pad proximate to the top and extending therefrom, such that the second element is positionable over the tube of the catheter positioned upon the first element, positioning the second element for engaging the first element, such that the hook and loop fastener stabilizes the catheter in the selected position.

12. The catheter and a catheter stabilizing device combination of claim 7, further including a sheet removably engaged to the adhesive, such that the sheet is positioned for preventing inadvertent adhesion of the adhesive, wherein the sheet is configured for removal from the adhesive, positioning the adhesive for adhesively engaging the pad to the abdominal skin of the user.

13. A catheter stabilizing device comprising:
a pad, the pad being substantially ovally shaped, the pad having a pair of cutouts extending arcuately and singly thereinto from opposed ends thereof, wherein the cutouts are configured for facilitating conforming of the pad to contours of an abdomen of a user;
an adhesive engaged to a first face of the pad, wherein the adhesive is configured for adhesively engaging the pad to abdominal skin of the user, such that the pad is engaged to the abdomen of the user;
a connector engaged to a second face of the pad and being configured for selectively connecting to a tube of a catheter, wherein the connector is configured for stabilizing the catheter in a selected position, the connector comprising a first element and a second element of a hook and loop fastener, the first element being engaged to the second face of the pad and extending from proximate to a bottom to proximate to a top of the pad, the second element being engaged to the pad proximate to the top and extending therefrom, wherein the second element is configured for positioning over the tube of a catheter positioned upon the first element, positioning the second element for engaging the first element, wherein the hook and loop fastener is configured for stabilizing the catheter in the selected position, the connector being one of a pair of connectors, each connector being positioned proximate to an inner limit of an associated cutout, wherein each connector is configured for selectively connecting to a respective site along the tube of the catheter, such that the tube extends loopedly across the second face of the pad; and
a sheet removably engaged to the adhesive, such that the sheet is positioned for preventing inadvertent adhesion of the adhesive, wherein the sheet is configured for removal from the adhesive, positioning the adhesive for adhesively engaging the pad to the abdominal skin of the user.
